(19) United States
(12) Patent Application Publication
(10) Pub. No.: US 2005/0266097 A1
(43) Pub. Date: Dec. 1, 2005

(54) Quatrepolymer Tablet

(75) Inventor: Daryl G. Meyer, Huron, OH (US)

Correspondence Address: DARYL MEYER;ENVIRO PURE INC., 2523 SCHEID ROAD, HURON, OH, 44839, US

(73) Assignee: Daryl G. Meyer, Huron, OH (US)

(21) Appl. No.: 10/250,048

(22) Filed: May 30, 2003

Related U.S. Application Data
(63) Continuation-in-part of application No. 10/064,991, filed on Sep. 6, 2002, now abandoned.

(51) Int. Cl. A61K 33/14; C02F 1/76

(52) U.S. Cl. 424/661; 210/756

(57) ABSTRACT

A compressed mixture of granular quatrepolymer compositions, quatrepolymer compositions and calcium hypochlorite and/or a hypochlorite-bromine additive for wastewater disinfection, or quatrepolymer compositions and a dechlorination agent and a wax binder selected from the group consisting of microcrystalline hydrocarbon wax and alkali metal salts of oxidized microcrystalline hydrocarbon wax. The mixture dissolves at a controlled rate, depending on the amount of wax binder used, in water thereby providing a source of available quatrepolymer compositions, quatrepolymer compositions and calcium hypochlorite or a hypochlorite—bromine additive for wastewater disinfection, or quatrepolymer compositions and a dechlorination agent over an extended period of time.

### CROSS REFERENCE TO RELATED APPLICATIONS

This is a Continuation In Part of patent application Ser. No. 10/064,991 first filed electronically on Sep. 06, 2002 (Sep. 6, 2002) at 15:07:38 EDT. with an additional claim to the original filing.

### SUMMARY OF INVENTION

This is a Continuation In Part of patent application Ser. No. 10/064,991 first filed electronically on Sep. 06, 2002 (Sep. 6, 2002) at 15:07:38 EDT. with an additional claim to the original filing. The present invention relates to quatrepolymer compositions, quatrepolymer compositions and calcium hypochlorite or a hypochlorite—bromine additive for wastewater disinfection and quatrepolymer compositions and a dechlorination additive such as sodium sulfite, ascorbic acid or sodium ascorbate to neutralize a hypochlorite or a hypochlorite—bromine additive and, more particularly, relates to a solid quatrepolymer composition, such as a tablet, which provides a continuous source of available quatrepolymer compositions, quatrepolymer compositions and calcium hypochlorite or a hypochlorite—bromine additive for wastewater disinfection, or quatrepolymer and a dechlorination agent such as sodium sulfite, ascorbic acid or sodium ascorbate all for enhancing and accelerating drainage of wastewater effluent over an extended period of time. The addition of granular quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate to wastewater effluent results in improved soil drainage by electrical charge neutralization of the soil particles. This activity increases soil permeability, porosity, percolation and capillary action.

Quatrepolymer compositions enjoy a portion of the market for available polymer compounds, aside from polymer itself, because it is a cheap and stable composition, which delivers all of its available polymer immediately on contact with water. Quatrepolymer compositions containing up to 65 weight percent of polycarboxylic anionic quatrepolymer and maleic sulfonated copolymer have been on the market for many years and are used primarily as a commercial soil conditioner and drainage agent, particularly in the residential, commercial, farming and nursery industry. As a source of available polymer, quatrepolymer compositions are a highly soluble material that dissolves rapidly in water.

For the treatment of wastewater effluent water, it is conventional to broadcast liquid or dry polymer periodically directly on or into the effluent water in the clarifier, filter device, polymer contact tank or pumping chamber in quantities sufficient to maintain the available polymer at or above the desired levels, e.g., from less than one (1) part per million to several parts per million of polymer for suspended solids removal. In an alternative method, tablets of quatrepolymer compositions, quatrepolymer compositions and a calcium hypochlorite or a hypochlorite—bromine additive for wastewater disinfection, or quatrepolymer compositions and a dechlorination agent such as sodium sulfite, ascorbic acid or sodium ascorbate are placed in the tablet feeder to provide continuous contact between the wastewater effluent water and the solid quatrepolymer composition. A further method used to treat wastewater effluent water is to add granular or tabletted quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate to a dispensing device in which the quatrepolymer composition is contacted with the water to be treated so that dissolution of the quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate is controlled to form a solution of the desired available quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate concentration. This concentrated solution is then added to the total body of wastewater effluent water to provide the desired available quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate concentration.

When added to water at room temperatures, quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate dissolve rapidly. Consequently, treatment of water, e.g., wastewater effluent water, is required almost daily to maintain a quantity of available quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate in the wastewater effluent for enhanced or accelerated drainage. A source of quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate, which provides a relatively constant source of available quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate over a prolonged period, e.g., 4-6 or 7 days, is a highly desirable feature for the consumer and ultimate user of quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate.

It has now been discovered that a compressed mixture of granular quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate and from about 0.5 to about 5 percent, basis the quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate, of a solid wax binder selected from the group consisting of microcrystalline hydrocarbon wax and alkali metal salts of oxidized microcrystalline hydrocarbon wax provides an article which, when placed in contact with water, dissolves more slowly than an article composed of binder-free compressed granular quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate. When such articles are placed in the tablet feeder of a wastewater treatment installation, dissolution of the quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate in the article is prolonged for from 4 to 6 days during which time the article provides a source of available quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate in amounts sufficient for enhancing and accelerating drainage, i.e., a reactive or accelerated amount.

### DETAILED DESCRIPTION

This is a Continuation In Part of patent application Ser. No. 10/064,991 first filed electronically on Sep. 06, 2002 (Sep. 6, 2002) at 15:07:38 EDT. with an additional claim to the original filing. Liquid quatrepolymer compositions are commercially available and generally contains from 45 to 65 percent by weight of polycarboxylic anionic quatrepolymer and maleic sulfonated copolymer. The remainder of the quatrepolymer article of commerce is typically composed of varying amounts of nitrogen, phosphate, potash, thiamine nitrate, inert ingredients and water. The aforementioned salts are incorporated into the commercial quatrepolymer composition product during its synthesis and manufacture. Water and inert ingredients may compose up to 27 percent, e.g., up to 55 percent of the quatrepolymer composition, but this should not be a limitation upon the scope of the invention.

In forming the article of the present invention, granular quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate are blended with the binder material and compacted with pressure into the shape desired, e.g., a tablet. The granular quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate material may exhibit a relatively broad particle size distribution. The latter physical property permits the smaller particles to fill in the spaces between the larger particles during compaction. It is preferred that the particles comprising the granular quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate be particulate rather than powdery in size since the size of the particles compacted is believed to influence the rate of dissolution of the compacted article, but this should not be a limitation upon the scope of the invention.

As a general guideline, the particles may have a principal size distribution between about minus 6 and plus 100 U.S. Sieve Series, i.e., the particles vary in size principally between about 0.13 inches (3.3 millimeters) and about 0.006 inches (0.15 millimeters). More commonly, the particles will have a principal size distribution between about minus 6 and plus 60 U.S. Sieve, but this should not be a limitation upon the scope of the invention.

Particularly suitable for use in producing articles of the present invention are granular quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate having a size distribution of minus 10, and plus 45 U.S. Sieve Series, i.e., the granules are principally between about 0.08 and 0.014 inches (1.98 and 0.35 millimeters), but this should not be a limitation upon the scope of the invention. Particles smaller than 60 U.S. Sieve present in the granular quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate represent a minor percentage, usually less than 2 percent, of the material charged to the compaction device but this should not be a limitation upon the scope of the invention.

The binders used to agglomerate the granular quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate in accordance with the present invention are microcrystalline hydrocarbon waxes. Microcrystalline wax consists of high molecular weight saturated aliphatic hydrocarbons, e.g., hydrocarbons containing about 48 carbon atoms. These waxes are available commercially and are produced by deoiling and cutting petrolatums or pipe still bottoms. The oxidized microcrystalline waxes are prepared by oxidizing the microcrystalline wax with air in the presence of a catalyst such as manganese or cobalt, but this should not be a limitation upon the scope of the invention.

The wax binder is used in the amounts of between about 0.5 and about 5.0 weight percent, basis the quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate, but this should not be a limitation upon the scope of the invention. Typically, the higher the amount of binder used within that range the longer is the dissolution time for the quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate of the present invention. Hence, varying the amount of binder in said compositions controls their rate of dissolution in water; such rate being adjustable with the amount of binder used. For wastewater effluents, binder levels of between about 0.5 and 2 weight percent are useful, but this should not be a limitation upon the scope of the invention. Such binder levels will provide dissolution times of between about 4 and 6 or 7 days. Preferably between about 0.75 and about 1.75, e.g., 1.5, weight percent of the aforesaid wax binder is used, but this should not be a limitation upon the scope of the invention. The microcrystalline waxes used herein as binders are available as finely divided powders and are available commercially as Petrolite, RTM series of microcrystalline hydrocarbon and modified hydrocarbon waxes, but this should not be a limitation upon the scope of the invention. These microcrystalline waxes having a melt point of between about 95.degree. C. and 105.degree. C., but this should not be a limitation upon the scope of the invention.

The articles of the present invention are prepared by dry blending granular quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate and the solid wax binder in a suitable blender and feeding the resulting mixture to conventional size-enlargement equipment such as a molding press, tableting press, pellet mill and screw extruder, but this should not be a limitation upon the scope of the invention.

To assist in releasing the compressed quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate article from the forming equipment, it may be convenient to incorporate a small amount of lubricant (mold release agent) with the granular quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate-microcrystalline hydrocarbon wax blend or to dust the molds of the forming equipment with such agents, but this should not be a limitation upon the scope of the invention. Typical lubricants that may be used include metal salts of fatty acids, e.g., fatty acids having at least 10 carbon atoms such as metal salts of stearic acid, but this should not be a limitation upon the scope of the invention. The cation of the metal salt should be compatible with quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate, e.g., sodium, magnesium, zinc and calcium, but this should not be a limitation upon the scope of the invention. The proportion of the lubricant utilized may be between about 0.1 and about 1.0 percent by weight, but this should not be a limitation upon the scope of the invention.

The compressed article of the present invention may be prepared in any convenient desired shape, e.g., a brick, briquette, triangle, pellet, tablet, etc., depending upon the intended use of the article. Preferably, the shape is that of a tablet but this should not be a limitation upon the scope of the invention. The compressed article of the present invention may typically have a mass of between about 250 and about 350, e.g., about 300, grams of which up to 96.5 percent, is the granular formulation, but this should not be a limitation upon the scope of the invention. The compressed article may be of a size that may be inserted readily into a tablet-feeding device used with wastewater treatment or dissolvers used to form concentrated solutions of quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate. In the case of a tablet, it is preferred that the diameter of such tablet be between about 3 inches (7.6 centimeters) and about 3.5 inches (8.9 centimeters), e.g., between about 3.125-3.25 inches (7.9-8.3 centimeters), and be about 1 to 2 inches (2.5-5.1 centimeters), e.g., 1.25 inches (3.2 centimeters) thick, but this should not be a limitation upon the scope of the invention. The compacted article preferably has a density of from about 1.6-2.3 grams per cubic centimeter (g/cc), e.g., 1.8-2.2 g/cc, more preferably about 2 g/cc, but this should not be a limitation upon the scope of the invention. Generally, the denser the compacted article, the slower the dissolution of the article.

Solid articles of compressed granular quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate prepared in accordance with the present invention will dissolve slowly over a period of between about 4 and 6 or 7 days, depending on the ultimate mass of the article and the amount of binder used, when placed in a conventional tablet feeder used in association with wastewater treatment and contacted with wastewater effluent. The slow dissolution of the aforesaid article thereby provides a source of available quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate for enhancing and accelerating drainage of the effluent over the period of time required to dissolve substantially all of the quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate in the article, e.g., a tablet. In a preferred embodiment, the solid article is prepared in a manner such that at least 10 weight percent of the original compressed quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate will remain after about 6 days of such use, but this should not be a limitation upon the scope of the invention. More preferably, the quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate provides a relatively constant source of quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate to the effluent water so as to maintain from less than one (1) to several parts per million of quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate therein, but this should not be a limitation upon the scope of the invention.

The present invention is more particularly described in the following examples, which are intended as illustrative only since numerous modifications, and variations therein will be apparent to those skilled in the art, but this should not be a limitation upon the scope of the invention.

### EXAMPLE 1

An eight quart V-blender was charged with 6.5 pounds of granular quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate having a size distribution of minus 6 and plus 100 U.S. Sieve Screen size and up to 65% available quatrepolymer composition. The desired level of additive was added to the quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate in the blender and covered with a second 6.5 of the same granular quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate. The blender was closed and ran for 45 minutes to blend the ingredients. The blend was charged to a Stokes R-tablet press having a die 2.5 inches (6.35 centimeters) in diameter and 1 inch (2.54 centimeters) in depth and tablets produced from the blend using a tableting pressure of 20 tons (18.1 metric tons). The additives and their amounts are listed in Table 1.

TABLE 1

| | | Tablet Wt, |
|---|---|---|
| | | % |
| Additive | Amount, Wt. | Grams .sup.e |
| 1. None | 0 | 170 |
| 2. Calcium Stearate | 1 | — |
| 3. Calcium Stearate | 2 | — |
| 4. Microcrystalline Wax .sup.a | 1.5 | 184 |
| 5. Modified Microcryatalline Wax .sup.b | 1.5 | 177 |
| 6. Synthetic Clay .sup.c | 2 | — |
| 7. Calcium Carbonate | 2 | — |
| 8. Chlorinated Paraffin .sup.d | 2 | — |

.sup.a Petrolite .RTM. X2010
.sup.b Petrolite .RTM. X5082
.sup.c Laponite .RTM. Hectorite
.sup.d Chlorowax .RTM. 70
.sup.e Average of 10 tablets

Dissolution rate of the tablets (except 2 and 3) was measured by placing one tablet in a feeder or skimmer basket mounted in a plastic pail through which water at room temperature (about 24.degree C.) was circulated from a 55 gallon (0.21 cubic meters) drum and monitoring the available quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate tablets level in the circulating water.

The control tablet was exhausted in one day as were the calcium carbonate, Chlorowax.RTM. 70, and Laponite. RTM. clay modified tablets. The calcium stearate modified tablets were brittle and of such poor quality with respect to physical integrity that their dissolution rates were not measured. The microcrystalline wax modified tablets had significantly slower dissolution rates-about 90 percent of the tablet being exhausted in about six days.

### EXAMPLE 2

Quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate tablets modified with 1.5 weight percent Petrolite. RTM X-2010 microcrystalline wax and having a tablet weight of about 292 grams were prepared from granular quatrepolymer compositions, quatrepolymer compositions and calcium hypochlorite or a hypochlorite—bromine additive for wastewater disinfection, or quatre polymer compositions and a dechlorination agent such as sodium sulfite, ascorbic acid or sodium ascorbate having a size distribution of minus 10 and plus 100 U.S. Sieve and up to 65 percent available quatrepolymer compositions in accordance with the procedure used in Example 1 except that a tableting pressure of 45 tons (40.7 metric tons) and a die having a diameter of 3.125 inches (7.94 centimeters) and a depth of 2 inches (5.1 centimeters) was used. About 98 percent of the granular product was in the range of minus 10 and plus 45 U.S. Sieve. The dissolution rate of such tablets was tested by the technique described in Example 1. The pH and temperature of the circulating solution for test A were maintained at about 7.5 and 24.degree. C. respectively. For test B, the pH and temperature were about 7.7 and 23.degree.-24.degree. C. Results for two such dissolution rate tests are tabulated in Table II.

TABLE 2

| Test A | | Test B | |
|---|---|---|---|
| | Ave. Avail. | | Ave. Avail. |
| Elapsed time, | Chlorine in addtion to | Elapsed time, | Chlorine in addition to |
| Hours | Quatrepolymer, ppm. | Hours | Quatrepolymer, ppm. |
| 0 | 33.3 | 0 | 19.8 |
| 24.65 | 383.0 | 24.65 | 358.1 |
| 49.01 | 485.8 | 49.0 | 446.8 |
| 89.54 | 581.5 | 89.5 | 595.7 |
| 115.0 | 691.5 | 115.0 | 691.5 |
| 137.5 | 762.4 | 137.4 | 744.7 |
| 162.3 | 783.7 | 162.2 | 751.8 |

The data of Table I and II show that microcrystalline wax-modified quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate tablets had significantly slower dissolution rates than unmodified tablets and provided quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate to the circulating aqueous medium for from about 6 to 7 days, depending on the total weight of the tablet.

Although the present process has been described with reference to specific details of certain embodiments thereof, it is not intended that such detail should be regarded as limitations upon the scope of the invention except as and to the extent that they are inclined in the accompanying claims or future filings relating to additional claims on the product, chemistry or methods of manufacture.

Additional information is available on USPTO Disclosure Documents 511808, 511810, 511811 and 511812 copies of which are include in the Figures Section.

1. a solid article comprising a compressed mixture of a granular quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate and from about 0.5 to about 5.0 weight percent of a solid wax binder selected from the group consisting of microcrystalline hydrocarbon wax and alkali metal salts of oxidized microcrystalline hydrocarbon wax, but this should not be a limitation upon the scope of the invention.

2. The article of claim 1 wherein the principal size distribution of the granular quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate is between minus 6 and plus 100 U.S. Sieve, but this should not be a limitation upon the scope of the invention.

3. The article of claim 2 wherein the binder is present in amounts of from 0.75 to 1.75 weight percent, but this should not be a limitation upon the scope of the invention.

4. The article of claim 3 wherein the binder is the potassium salt of oxidized microcrystalline hydrocarbon wax, but this should not be a limitation upon the scope of the invention.

5. The article of claim 3 wherein the mass of the article is from 250 to 350 grams, but his should not be a limitation upon the scope of the invention.

6. The article of claim 5 wherein the shape of the article is in the form of a tablet having a diameter of from about 3 to about 3.5 inches, but this should not be a limitation upon the scope of the invention.

7. The article of claim 1 wherein at least 10 weight percent of the original mass of compressed quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate remains after 6 days of contact with water, but this should not be a limitation upon the scope of the invention.

8. The article of claim 1 wherein the granular quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate is up to 65 weight percent polycarboxylic anionic quatrepolymer, but this should not be a limitation upon the scope of the invention.

9. The article of claim 2 wherein the principal size distribution of the granular quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate is between minus 10 and plus 45 U.S. Sieve, but this should not be a limitation upon the scope of the invention.

10. The article of claim 9 wherein the binder is present in amounts of from 0.75 to 1.75 weight percent, but this should not be a limitation upon the scope of the invention.

11. The article of claim 10 wherein the density of the article is between about 1.6 and 2.3 grams per cubic centimeter, but this should not be a limitation upon the scope of the invention.

12. The article of claim 10 wherein the principal size distribution of the granular quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate is between minus 6 and plus 60 U.S. Sieve, but this should not be a limitation upon the scope of the invention.

13. The article of claim 1 wherein the addition of granular quatrepolymer compositions, quatrepolymer compositions and calcium hypochlorite or a hypochlorite—bromine additive for wastewater disinfection, or quatrepolymer compositions and a dechlorination agent such as sodium sulfite, ascorbic acid or sodium ascorbate to wastewater effluent results in improved soil drainage by electrical charge neutralization of the soil particles. This activity increases soil permeability, porosity, percolation and capillary action.
